(19) United States
(12) Patent Application Publication
(10) Pub. No.: US 2022/0096495 A1
(43) Pub. Date: Mar. 31, 2022

(54) TREATMENT AND PREVENTION OF INTESTINAL INFLAMMATORY DISEASES WITH A BILE ACID DERIVATIVE

(71) Applicant: Intercept Pharmaceuticals, Inc., New York, NY (US)

(72) Inventors: Laurens CEULEMANS, Tremelo (BE); Emilio CANOVAI, Hoogstraten (BE)

(21) Appl. No.: 17/428,029

(22) PCT Filed: Feb. 3, 2020
(86) PCT No.: PCT/US2020/016329

§ 371 Date: Aug. 3, 2021

Related U.S. Application Data
(60) Provisional application No. 62/801,066, filed on Feb. 4, 2019.

(51) Int. Cl. A61K 31/57; A61P 9/10; A61P 1/00

(52) U.S. Cl. CPC A61K 31/57; A61P 1/00; A61P 9/10

(57) ABSTRACT

The present application relates to methods of treating or preventing an intestinal inflammatory disease or condition (e.g., intestinal ischemia reperfusion injury) in a subject in need thereof, comprising administering a therapeutically effective amount of a compound of the application.

### BACKGROUND

The farnesoid X receptor (FXR), a member of the nuclear receptor family, is abundantly expressed in the ileum, where it exerts an enteroprotective role as a key regulator of intestinal innate immunity and homeostasis, as shown in pre-clinical models of inflammatory bowel disease. TGR5 is a G-protein-coupled bile activated receptor. Both are abundantly expressed in the gastrointestinal tract. In pre-clinical models, both have been shown to reduce inflammation and improve epithelial permeability. Pretreatment with obeticholic acid (OCA), an FXR-agonist, improved survival in a rodent model of intestinal IM, preserves the gut barrier function and suppresses inflammation. (Ceulemans, et al., PLoS One (2017) 12(1): e0169331). Based on these results, FXR appears to be a promising target for diseases or conditions and various pathologies associated gut barrier function and intestinal inflammation.

Intestinal ischemia reperfusion injury (IRI) is characterized by hyperpermeability, bacterial translocation and inflammation. It was found that intestinal TM was associated with high mortality (90%); loss of intestinal integrity (structurally and functionally); increased endotoxin translocation and pro-inflammatory cytokine production; and inhibition of autophagy. Ischemia reperfusion injury (TM) occurs inevitably during intestinal transplantation and after intestinal infarction. Intestinal grafts are especially susceptible to IRI which leads to loss of villi, resulting in systemic translocation which contributes to poorer outcomes.

There is a need for therapies for the treatment and prevention of the intestinal inflammatory diseases or conditions and pathologies associated gut barrier function and intestinal inflammation (e.g. IM). The present application addresses the need.

### SUMMARY

The present application relates to a method of treating or preventing an intestinal inflammatory disease or condition (e.g., ischemia reperfusion injury or IM), in a subject in need thereof, comprising administering to the subject a therapeutically effective amount of a compound of Formula A:

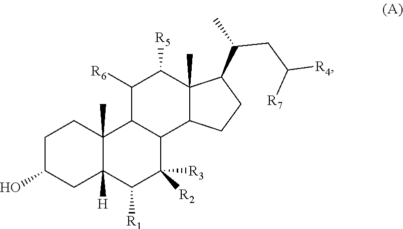

or a pharmaceutically acceptable salt or amino acid conjugate thereof, wherein:

R₁ is C₁-C₆ alkyl;

R₂, R₃, R₅, and R₆ are each independently H or OH;

R₄ is CO₂H or OSO₃H; and

R₇ is H or C₁-C₆ alkyl.

The present application also relates to a compound of Formula A, or a pharmaceutically acceptable salt or amino acid conjugate thereof, for treating or preventing intestinal inflammatory disease or condition, such as intestinal ischemia reperfusion injury (IRI), in a subject in need thereof.

The present application also relates to a compound of Formula A, or a pharmaceutically acceptable salt or amino acid conjugate thereof, for use in the manufacture of a medicament for the treatment or prevention of intestinal inflammatory disease or condition (e.g., intestinal ischemia reperfusion injury or IRI), in a subject in need thereof.

The present application also relates to use of a compound of Formula A, or a pharmaceutically acceptable salt or amino acid conjugate thereof, in the manufacture of a medicament for the treatment or prevention of intestinal inflammatory disease or condition (e.g., intestinal ischemia reperfusion injury or IRI), in a subject in need thereof.

In one embodiment, a compound of Formula A is Compound 1:

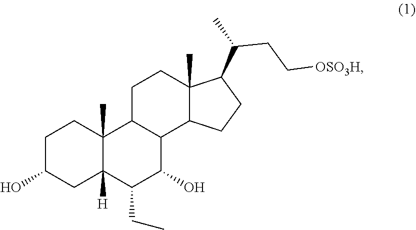

or a pharmaceutically acceptable salt thereof.

In one embodiment, a pharmaceutically acceptable salt of Compound 1 is the sodium salt of Compound 1 (i.e., Compound 1-Na). In another embodiment, a pharmaceutically acceptable salt of Compound 1 is the triethylammonium salt of Compound 1 (i.e., Compound 1-TEA).

Unless otherwise defined, all technical and scientific terms used herein have the same meaning as commonly understood by one of ordinary skill in the art to which this application belongs. In the case of conflict, the present specification, including definitions, will control. In the specification, the singular forms also include the plural unless the context clearly dictates otherwise. Although methods and materials similar or equivalent to those described herein can be used in the practice or testing of the present application, suitable methods and materials are described below. All publications, patent applications, patents, and other references mentioned herein are incorporated by reference. The references cited herein are not admitted to be prior art. In the case of conflict, the present specification, including definitions, will control. In addition, the materials, methods, and examples are illustrative only and are not intended to be limiting. Other features and advantages of the application will be apparent from the following detailed description and claims.

### BRIEF DESCRIPTION OF THE DRAWINGS

FIG. 1 shows the histological damage score (Park-Chiu) of treated (with Compound 1) and untreated rats; compared to sham subjects.

FIG. 2 is a graph showing electrical resistance (TEER) measurements in rats pre-treated with either Compound 1 (INT-767) or vehicle 15 min after start of ischemia.

FIG. 3 is a graph showing FD20 permeability measurements in rats pre-treated with either Compound 1 (INT-767) or vehicle 15 min after start of ischemia.

### DETAILED DESCRIPTION

The present application is based at least in part on the discovery that a compound of Formula A or a pharmaceutically acceptable salt or amino acid conjugate thereof is effective in preventing or treating intestinal inflammatory disease or condition (e.g., intestinal ischemia reperfusion injury or IRI).

Accordingly, the present application relates to a method of treating or preventing an intestinal inflammatory disease or condition (e.g., IRI), in a subject in need thereof, comprising administering to the subject a therapeutically effective amount of a compound of Formula A:

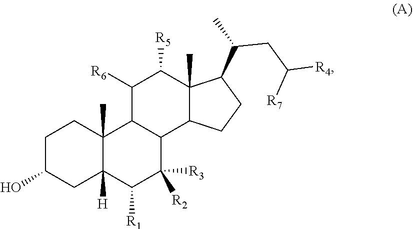

or a pharmaceutically acceptable salt or amino acid conjugate thereof, wherein:

R₁ is C₁-C₆ alkyl;

R₂, R₃, R₅, and R₆ are each independently H or OH;

R₄ is CO₂H or OSO₃H; and

R₇ is H or C₁-C₆ alkyl.

In one embodiment, a compound of Formula A is of Formula B or Formula C:

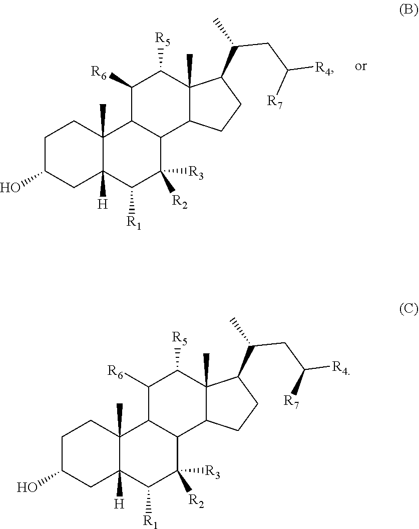

For any of Formula A, B, or C, R₁, R₂, R₃, R₄, R₅, R₆, and R₇ can be selected from the groups, and combined, where applicable, as described below.

In one embodiment, R₁ is C₁-C₆ alkyl selected from methyl, ethyl, n-propyl, i-propyl, n-butyl, i-butyl, t-butyl, pentyl, and hexyl. In one embodiment, R₁ is C₁-C₄ alkyl selected from methyl, ethyl, n-propyl, i-propyl, n-butyl, i-butyl, and t-butyl. In one embodiment, R₁ is methyl, ethyl, n-propyl, or i-propyl. In one embodiment, R₁ is methyl or ethyl. In one embodiment, R₁ is methyl. In one embodiment, R₁ is ethyl.

In one embodiment, R₂ is H and R₃ is OH. In one embodiment, R₃ is H and R₂ is OH.

In one embodiment, R₅ is H. In one embodiment, R₅ is OH.

In one embodiment, R₂ is H, R₃ is OH, and R₅ is H. In one embodiment, R₂ is H, R₃ is OH, and R₅ is OH.

In one embodiment, R₇ is H. In one embodiment, R₇ is C₁-C₆ alkyl selected from methyl, ethyl, n-propyl, i-propyl, n-butyl, i-butyl, t-butyl, pentyl, and hexyl. In one embodiment, R₇ is C₁-C₄ alkyl selected from methyl, ethyl, n-propyl, i-propyl, n-butyl, butyl, and t-butyl. In one embodiment, R₇ is methyl, ethyl, n-propyl, or i-propyl. In one embodiment, R₇ is methyl or ethyl. In one embodiment, R₇ is methyl.

In one embodiment, R₂ is H, R₃ is OH, and R₇ is H. In one embodiment, R₂ is H, R₃ is OH, and R₇ is methyl.

In one embodiment, R₂ is H, R₃ is OH, R₅ is H, and R₇ is H. In one embodiment, R₂ is H, R₃ is OH, R₅ is OH, and R₇ is methyl.

In one embodiment, R₆ is H. In one embodiment, R₆ is OH.

In one embodiment, R₂ is H, R₃ is OH, and R₆ is H. In one embodiment, R₂ is H, R₃ is OH, and R₆ is OH.

In one embodiment, R₂ is H, R₃ is OH, R₅ is H, and R₆ is H. In one embodiment, R₂ is H, R₃ is OH, R₅ is OH, and R₆ is OH. In one embodiment, R₂ is H, R₃ is OH, R₅ is OH, and R₆ is H. In one embodiment, R₂ is H, R₃ is OH, R₅ is H, and R₆ is OH.

In one embodiment, R₄ is CO₂H. In one embodiment, R₄ is OSO₃H.

In one embodiment, R₂ is H, R₃ is OH, R₄ is CO₂H, and R₅ is H. In one embodiment, R₂ is H, R₃ is OH, R₄ is OSO₃H, and R₅ is H. In one embodiment, R₂ is H, R₃ is OH, R₄ is CO₂H, and R₅ is OH. In one embodiment, R₂ is H, R₃ is OH, R₄ is OSO₃H, and R₅ is OH. In a further embodiment, R₁ is ethyl.

In one embodiment, R₂ is H, R₃ is OH, R₄ is CO₂H, and R₅ is OH. In one embodiment, R₂ is H, R₃ is OH, R₄ is CO₂H, R₅ is OH, and R₇ is methyl. In a further embodiment, R₁ is ethyl.

In one embodiment, R₂ is H, R₃ is OH, R₄ is CO₂H, and R₅ is H. In one embodiment, R₂ is H, R₃ is OH, R₄ is CO₂H, R₅ is H, and R₆ is OH. In a further embodiment, R₁ is ethyl.

In one embodiment, a compound of Formula A is Compound 1:

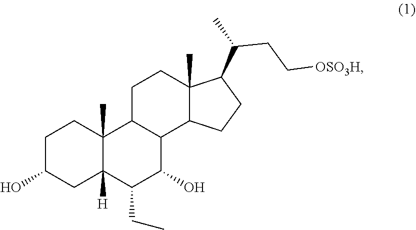

or a pharmaceutically acceptable salt thereof.

In one embodiment, a compound of Formula A is Compound 2:

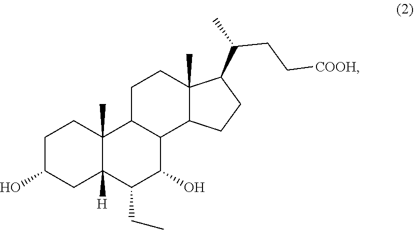 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]

or a pharmaceutically acceptable salt or amino acid conjugate thereof.

In one embodiment, a compound of Formula A is Compound 3:

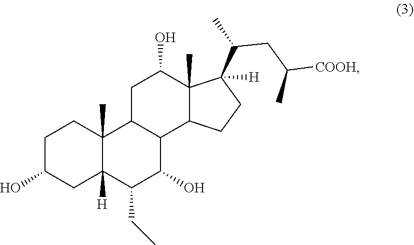 [SMILES: CC[C@H]1[C@@H](O)C2C3CC[C@H]([C@H](C)C[C@H](C)C(=O)O)[C@@]3(C)[C@@H](O)CC2[C@@]2(C)CC[C@@H](O)C[C@@H]12]

or a pharmaceutically acceptable salt or amino acid conjugate thereof.

In one embodiment, a compound of Formula A is Compound 4:

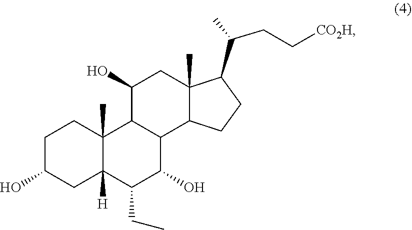 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

or a pharmaceutically acceptable salt or amino acid conjugate thereof.

In one embodiment, a pharmaceutically acceptable salt of Compound 1 is the sodium salt of Compound 1 (i.e., Compound 1-Na). In yet another embodiment, a pharmaceutically acceptable salt of Compound 1 is the triethylammonium salt of Compound 1 (i.e., Compound 1-TEA).

As used herein, the term “Compound 1” refers to

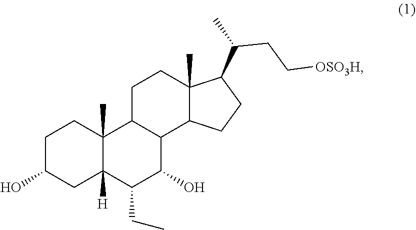

which is also known as 6α-ethyl-3α,7α,23-trihydroxy-24-nor-5β-cholan-23-hydrogen sulphate. “Compound 1-Na” or “1-Na” which is also known as INT-767 or 6α-ethyl-3α,7α,23-trihydroxy-24-nor-5β-cholan-23-sulphate sodium” are used interchangeably and refer to the sodium salt of Compound 1. As used herein, “Compound 1-TEA” or “1-TEA” is used interchangeably and refer to the triethylammonium salt of Compound 1 The structures of Compound 1-Na and Compound 1-TEA are provided below.

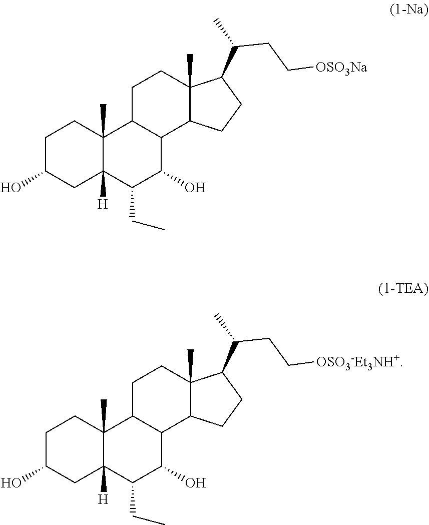

The phrase a “compound of the application” or “compound of the present application” as used herein encompasses a compound of Formula A, Formula B, or Formula. C, Compound 1, 1-Na, 1-TEA, Compound 2, Compound 3, or Compound 4, or a pharmaceutically acceptable salt or amino acid conjugate thereof.

As used herein, the term “amino acid conjugate” refers to a conjugate of the compound of the present application with any suitable amino acid. For example, such a suitable amino acid conjugate of a compound of the present application will have the added advantage of enhanced integrity in bile or intestinal fluids. Suitable amino acids include but are not limited glycine (—NHCH₂CO₂H), taurine (—NH(CH₂)₂SO₃H), and sarcosine (—N(CH₃)CH₂CO₂H). Thus, the present application encompasses the glycine, taurine, and sarcosine conjugates of the compound of the present application (e.g., Compound 2).

As used herein, FXR refers to Farnesoid X Receptor, which is a member of the nuclear receptor family of ligand-activated transcription factors that includes receptors for the steroid, retinoid, and thyroid hormones. FXR binds to DNA as a heterodimer with the 9-cis retinoic acid receptor (RXR).

As used herein, TGR5 refers to a G-protein-coupled receptor that is responsive to bile acids (BAs).

As used herein, a “subject in need thereof” is a subject having an intestinal inflammatory disease or condition (e.g., IRI) against which a compound of the application is effective, or a subject having an increased risk of developing intestinal inflammatory disease or condition against which a compound of the application is effective relative to the population at large. A “subject” includes a mammal. The mammal can be any mammal, e.g., a human, primate, bird, mouse, rat, fowl, dog, cat, cow, horse, goat, camel, sheep or a pig. Particularly, the mammal is a human.

The term “treating” as used herein refers to any indicia of success in the treatment or amelioration of any of the diseases, disorders, or conditions described herein. Treating can include, for example, reducing or alleviating the severity of one or more symptoms of any of the diseases, disorders, or conditions described herein, or it can include reducing the frequency with which symptoms of any of the diseases, disorders, or conditions described herein are experienced by a patient. “Treating” can also refer to reducing or eliminating any of the diseases, disorders, or conditions described herein of a part of the body, such as a cell, tissue or bodily fluid.

As used herein, the term “preventing” refers to the partial or complete prevention of any of the diseases, disorders, or conditions described herein in an individual or in a population, or in a part of the body, such as a cell, tissue or bodily fluid. The term “prevention” does not establish a requirement for complete prevention of a disease, disorder, or condition in the entirety of the treated population of individuals or cells, tissues, or fluids of individuals.

The term “treat or prevent” is used herein to refer to a method that results in some level of treatment or amelioration of any of the diseases, disorders, or conditions described herein, and contemplates a range of results directed to that end, including but not restricted to prevention of any of the diseases, disorders, or conditions described herein entirely.

As used herein, “disease or condition” refers to various diseases, disorders or conditions associated intestinal inflammation and leading to intestinal and liver pathologies. For example, intestinal dysbiosis and bacterial translocation contribute to the inflammatory pathways involved in development of NASH (article is being prepared for publication). Changes in bile acids signaling have been demonstrated to modulate intestinal microbiota and consequently intestinal and liver pathologies. Clinical study (FLINT) demonstrated that OCA treatment improves histological and immune features in patients with NASH (Neuschwander-Tetri et al, Lancet, 2015). Intestinal and liver pathologies include, but are not limited to loss of intestinal integrity, bacterial translocation, increased permeability, as intestinal transplantation, sepsis, cirrhosis, NASH.

As used herein, “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have met the required standards of toxicological and manufacturing testing and/or are included on the Inactive Ingredient Guide prepared by the U.S. Food and Drug administration.

A “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The phrase “therapeutically effective amount” as used herein refers to an effective amount comprising an amount sufficient to treat a disease, disorder, or condition described herein or to prevent or delay a disease, disorder, or condition described herein. In some embodiments, an effective amount is an amount sufficient to delay the development of the disease, disorder, or condition. In some embodiments, an effective amount is an amount sufficient to prevent or delay recurrence. An effective amount can be administered in one or more administrations.

A therapeutically effective amount can be estimated initially either in cell culture assays or animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred.

The term “regimen” as used herein refers to a protocol for dosing and/or timing the administration a compound of the application. A regimen can include periods of active administration and periods of rest as known in the art. Active administration periods include administration of a compound of the application in a defined course of time, including, for example, the number of and timing of dosages of the compositions. In some regimens, one or more rest periods can be included where no compound is actively administered, and in certain instances, includes time periods where the efficacy of such compounds can be minimal.

In one embodiment, a compound of the present application is administered once daily, twice daily, three times daily, once every 6 hours, or once every 4 hours. In one embodiment, a compound of the present application is administered for one day, two days, three days, four days, five days, six days, or seven days a week. In one embodiment, a compound of the present application is not administered every day of the week. In one embodiment, a compound of the present application is administered every other day, once every three days, once every four days, once every five days, once every six days, or once every seven days.

In one embodiment, a compound of the present application is administered for a period of one week, two weeks, three weeks, four weeks, six weeks, two months, three months, four months, six months, or more. In one embodiment, the period in which a compound of the present application is administered comprises one or more segments (e.g., one or more days, one or more weeks, or one or more months) during which the compound is not administered. In one embodiment, the one or more segments during which the compound is not administered are preceded by and followed by administration of the compound.

As used herein, “combination therapy” means that a compound of the application can be administered in conjunction with another therapeutic agent. “In conjunction with” refers to administration of one treatment modality in addition to another treatment modality, such as administration of a compound of the application as described herein in addition to administration of another therapeutic agent to the same subject. As such, “in conjunction with” refers to administration of one treatment modality before, during, or after delivery of a second treatment modality to the subject.

The term “about” as used herein when referring to a measurable value such as an amount, a temporal duration, and the like, is meant to encompass variations of ±20% or ±10%, in some embodiments ±5%, in some embodiments ±1%, and in some embodiments ±0.1% from the specified value, as such variations are appropriate to practice the disclosed methods or to make and used the disclosed compounds and in the claimed methods.

Unless specified or the context dictates otherwise, a “pharmaceutical composition” or “pharmaceutical formulation” is used interchangeably and refers to a formulation containing a compound of the present application in a form suitable for administration to a subject. In one embodiment, the pharmaceutical composition is in bulk or in unit dosage form. It can be advantageous to formulate compositions in dosage unit form for ease of administration and uniformity of dosage. The specification for the dosage unit forms is dictated by and directly dependent on the unique characteristics of the active reagent and the particular therapeutic effect to be achieved. The unit dosage form is any of a variety of forms, including, for example, a capsule, an IV bag, a tablet, a single pump on an aerosol inhaler, or a vial.

Possible formulations include those suitable for oral, sublingual, buccal, parenteral (e.g., subcutaneous, intramuscular, or intravenous), rectal, topical including transdermal, intranasal, and inhalation administration. Most suitable means of administration for a particular patient will depend on the nature and severity of the disease being treated, the nature of the therapy being used, and the nature of the active compound.

Formulations suitable for oral administration may be provided as discrete units, such as tablets, capsules, cachets, lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising a compound of the application and typically a flavored base, such as sugar and acacia or tragacanth and pastilles comprising the active compound in an inert base, such as gelatin and glycerin or sucrose acacia.

Formulations suitable for parenteral administration typically comprise sterile aqueous solutions containing a predetermined concentration of the active compound; the solution may be isotonic with the blood of the intended recipient. Additional formulations suitable for parenteral administration include formulations containing physiologically suitable co-solvents and/or complexing agents such as surfactants and cyclodextrins. Oil-in-water emulsions are also suitable formulations for parenteral formulations. Although such solutions may be administered intravenously, they may also be administered by subcutaneous or intramuscular injection.

Formulations suitable for rectal administration may be provided as unit-dose suppositories comprising a compound of the application in one or more solid carriers forming the suppository base, for example, cocoa butter.

Formulations suitable for topical or intranasal application include ointments, creams, lotions, pastes, gels, sprays, aerosols, and oils. Suitable carriers for such formulations include petroleum jelly, lanolin, polyethyleneglycols, alcohols, and combinations thereof.

Oral formulations generally include an inert diluent or an edible pharmaceutically acceptable carrier. They can be enclosed in gelatin capsules or compressed into tablets. For the purpose of oral administration, the active ingredient can be incorporated with excipients and used in the form of tablets, troches, or capsules. Oral formulations can also be prepared using a fluid carrier for use as a mouthwash, wherein the active ingredient in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes®; a glidant such as colloidal silicon dioxide; a sweetening agent such as sucrose or saccharin; or a flavoring agent such as peppermint, methyl salicylate, or orange flavoring. Pharmaceutically compatible diluents may also include starch, dextrin, sucrose, glucose, lactose, mannitol, sorbitol, xylitol, microcrystalline cellulose, calcium sulfate, calcium hydrogen phosphate, calcium carbonate, and the like. Pharmaceutically compatible wetting agents included water, ethanol, isopropanol, and the like. Pharmaceutically compatible binders may also include starch pulp, dextrin, syrup, honey, glucose solution, microcrystalline cellulose, mucilage of arabic gum, gelatin mucilage, sodium hydroxymethylcellulose, methylcellulose, hydroxypropylmethylcellulose, ethyl cellulose, acrylic resin, carbomer, polyvinyl pyrrolidone, polyethylene glycol, and the like. Pharmaceutically compatible disintegrants may also include dry starch, microcrystalline cellulose, low-substituted hydroxypropylcellulose, cross-linked polyvinylpyrrolidone, croscarmellose sodium, sodium carboxymethyl starch, sodium bicarbonate and citric acid, polyoxyethylene sorbitol fatty acid esters, sodium dodecyl sulfonate and the like. Pharmaceutically compatible lubricants and glidants may also include talc powder, silica, stearate, tartaric acid, liquid paraffin, polyethylene glycol, and the like.

Pharmaceutical formulations suitable for injectable use (e.g., intravenous, intramuscular) include sterile aqueous solutions (where water soluble), dispersions/suspensions, and sterile powders for the extemporaneous preparation of sterile injectable solutions or dispersions. Suitable carriers include physiological saline, bacteriostatic water, Cremophor EL™ (BASF, Parsippany, N.J.) or phosphate buffered saline (PBS). In some embodiment, the carrier or vehicle can be methylcellulose. The carriers can also be a solvent or dispersion medium containing, for example, water, ethanol, polyol (for example, glycerol, propylene glycol, and liquid polyethylene glycol, and the like), and suitable mixtures thereof. The composition must be sterile and should be fluid to the extent that easy syringeability exists. It must be stable under the conditions of manufacture and storage and must be preserved against contaminating by microorganisms such as bacteria and fungi. The proper fluidity can be maintained, for example, by the use of agents such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal, and the like. In many cases, it will include isotonic agents, for example, sugars, polyalcohols such as mannitol, sorbitol, sodium chloride in the composition. Other excipients include, but are not limited to, antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Prolonged absorption of the injectable compositions can be brought about by including in the composition an agent which delays absorption, for example, aluminum monostearate and gelatin. The preparation can be enclosed in ampoules, disposable syringes or multiple dose vials made of glass or plastic.

Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Formulations of the application may be prepared by any suitable method, typically by uniformly and intimately admixing a compound of the application with liquids or finely divided solid carriers or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by molding an intimate mixture of powdered active ingredient and inert liquid diluent. Suitable formulations for administration by inhalation include fine particle dusts or mists which may be generated by means of various types of metered dose pressurized aerosols, nebulizers, or insufflators.

In addition to the ingredients specifically mentioned above, the formulations of the present application may include other agents known to those skilled in the art of pharmacy, having regard for the type of formulation in issue. For example, formulations suitable for oral administration may include flavoring agents and formulations suitable for intranasal administration may include perfumes. In one embodiment, the pharmaceutical composition comprises a compound of the present application or a pharmaceutically acceptable amino acid conjugate or salt thereof in the amount of 0.1-1500 mg, 0.2-1200 mg, 0.3-1000 mg, 0.4-800 mg, 0.5-600 mg, 0.6-500 mg, 0.7-400 mg, 0.8-300 mg, 1-200 mg, 1-100 mg, 1-50 mg, 1-30 mg, 4-26 mg, or 5-25 mg. In one embodiment, the pharmaceutical composition comprises a compound of the present application or a pharmaceutically acceptable amino acid conjugate or salt thereof in the amount of 5-25 mg. In one embodiment, the pharmaceutical composition comprises a compound of the present application or a pharmaceutically acceptable amino acid conjugate or salt thereof in the amount of 1-5 mg, 5-10 mg, 10-15 mg, 15-20 mg, 20-25 mg. In one embodiment, the pharmaceutical composition comprises a compound of the present application or a pharmaceutically acceptable amino acid conjugate or salt thereof in the amount of about 1 mg, about 5 mg, about 10 mg, about 15 mg, about 20 mg, and about 25 mg.

In one embodiment, a pharmaceutical composition is administered in a dosage form which comprises a compound of the application in a daily total amount of less than 10 mg/kg, preferably less than 5 mg/kg, such as, for example 0.1-5.0 mg/kg, preferably 0.5-4.5 mg/kg, preferably 1.0-4.0 mg/kg, preferably 1.2-3.5 mg/kg, preferably 1.4-3.0 mg/kg, preferably 1.5-2.5 mg/kg, preferably 1.6-2.4 mg/kg.

Compounds and compositions of the application can be administered in therapeutically effective amounts in a combination therapy with one or more therapeutic agents (pharmaceutical combinations) or modalities. Where the compounds of the application are administered in conjunction with other therapies, dosages of the co-administered compounds will of course vary depending on the type of co-drug employed, on the specific drug employed, on the condition being treated and so forth. For example, synergistic effects can occur with substances.

Combination therapy includes the administration of the subject compounds in further combination with one or more other biologically active ingredients (such as, but not limited to, a FXR agonist, a TGR5 agonist, a second compound of Formula A (a second and different compound of Formula A) and non-drug therapies (such as, but not limited to, surgery or dietary treatment, gut microbiome species, etc.). For instance, the compounds of the application can be used in combination with other pharmaceutically active compounds, preferably compounds that are able to enhance the effect of the compounds of the application. The compounds of the application can be administered simultaneously (as a single preparation or separate preparation) or sequentially to the other drug therapy or treatment modality. In general, a combination therapy envisions administration of two or more drugs during a single cycle or course of therapy. In another aspect of the application, the compounds may be administered in combination with one or more separate pharmaceutical agents, e.g., a chemotherapeutic agent, an immunotherapeutic agent, or an adjunctive therapeutic agent.

In some embodiments, the compounds of the application can be used in regulating the gut microbiome by inhibiting bacterial growth. The observed interaction between bile acids and the human small intestinal microbiome suggested opportunities for microbiome biomarker discovery as well as novel modalities to engineer the human microbiome via FXR activation. (Friedman, et al. FXR-Dependent Modulation of the Human Small Intestinal Microbiome by the Bile Acid Derivative Obeticholic Acid. Gastroenterology. 2018 December; 155(6):1741-1752). In one of the embodiments, an additional biologically active ingredient is one or more gut microbiome species. In one of the embodiments, the present application relates to a method of treating or preventing an intestinal inflammatory disease or condition, comprising administering to a subject in need thereof Compound of formula A, or a pharmaceutically acceptable amino acid conjugate or salt thereof, and one or more gut microbiome species. The present application also relates to use of Compound of formula A, or a pharmaceutically acceptable amino acid conjugate or salt thereof, in combination with one or more gut microbiome species, in treating or preventing an intestinal inflammatory disease or condition. In one embodiment, the present application relates to a method of treating. In one embodiment, the present application relates to a method of preventing. In one embodiment the present application relates to a method of treating or preventing an intestinal inflammatory disease or condition, comprising administering to a subject in need thereof Compound of formula 1, 2, 3, or 4, or a pharmaceutically acceptable amino acid conjugate or salt thereof, and one or more gut microbiome species.

The present application also relates to use of combinational therapy of Compound of formula A, or a pharmaceutically acceptable amino acid conjugate or salt thereof, with one or more gut microbiome species in the manufacture of a medicament for treating or preventing an intestinal inflammatory disease or condition.

In some embodiments, the intestinal inflammatory diseases or conditions include, but are not limited to intestinal ischemia reperfusion injury (IRI), loss of intestinal integrity, bacterial translocation, increased permeability, and intestinal transplantation.

In one embodiment, the intestinal inflammatory disease or condition is modulated by FXR. In one embodiment, the intestinal inflammatory disease or condition can be decreased by stimulating the FXR receptor to inhibit pro-inflammatory cytokine release and reduce intestinal permeability. In one embodiment, the intestinal inflammatory disease or condition is modulated by TGR5. In one embodiment, the intestinal inflammatory disease or condition can be decreased by stimulating the TGR5 receptor to inhibit monocytes from producing pro-inflammatory cytokines and stimulating intestinal recovery through GLP-1 upregulation. In one embodiment, the intestinal inflammatory disease or condition is modulated by FXR and TGR5. In one embodiment, the intestinal inflammatory disease or condition can be decreased by stimulating the FXR receptor to inhibit pro-inflammatory cytokine release and reduce intestinal permeability and/or by stimulating the TGR5 receptor to inhibit monocytes from producing pro-inflammatory cytokines and stimulating intestinal recovery through GLP-1 upregulation.

In one embodiment, the intestinal inflammatory disease or condition is loss of intestinal integrity. In one embodiment, the intestinal inflammatory disease or condition is bacterial translocation. In one embodiment, the intestinal inflammatory disease or condition is increased permeability. In one embodiment, the intestinal inflammatory disease or condition is intestinal transplantation. In one embodiment, the intestinal inflammatory disease or condition is intestinal ischemia reperfusion injury (IRI).

In one embodiment, intestinal ischemia reperfusion injury (IRI) is modulated by FXR. In one embodiment, IRI is decreased by stimulating the FXR receptor to inhibit pro-inflammatory cytokine release and reduce intestinal permeability. In one embodiment, IRI is modulated by TRG5. In one embodiment, IRI is decreased by stimulating the TGR5 receptor to inhibit monocytes from producing pro-inflammatory cytokines and stimulating intestinal recovery through GLP-1 upregulation. In one embodiment IRI is modulated by FXR and TGR5. In one embodiment, IRI is decreased by stimulating the FXR receptor to inhibit pro-inflammatory cytokine release and reduce intestinal permeability and by stimulating the TGR5 receptor to inhibit monocytes from producing pro-inflammatory cytokines and stimulating intestinal recovery through GLP-1 upregulation.

In one of the embodiments, the present application relates to a method of reducing intestinal permeability, comprising administering to a subject in need thereof Compound of formula A, or a pharmaceutically acceptable amino acid conjugate or salt thereof. In one of the embodiments, the present application relates to a method of inhibiting pro-inflammatory cytokine release, comprising administering to a subject in need thereof Compound of formula A, or a pharmaceutically acceptable amino acid conjugate or salt thereof. In one of the embodiments, the present application relates to a method of reducing intestinal permeability and inhibiting pro-inflammatory cytokine release, comprising administering to a subject in need thereof Compound of formula A, or a pharmaceutically acceptable amino acid conjugate or salt thereof.

In one of the embodiments, the present application relates to a method of inhibiting monocytes from producing pro-inflammatory cytokines, comprising administering to a subject in need thereof Compound of formula A, or a pharmaceutically acceptable amino acid conjugate or salt thereof. In one of the embodiments, the present application relates to a method of stimulating intestinal recovery through GLP-1 upregulation, comprising administering to a subject in need thereof Compound of formula A, or a pharmaceutically acceptable amino acid conjugate or salt thereof. In one of the embodiments, the present application relates to a method of inhibiting monocytes from producing pro-inflammatory cytokines and stimulating intestinal recovery through GLP-1 upregulation, comprising administering to a subject in need thereof Compound of formula A, or a pharmaceutically acceptable amino acid conjugate or salt thereof.

In one embodiment, the present application relates to a method of inhibiting pro-inflammatory cytokine release, reducing intestinal permeability, inhibiting monocytes from producing pro-inflammatory cytokines and stimulating intestinal recovery through GLP-1 upregulation, comprising administering to a subject in need thereof Compound of formula A or a pharmaceutically acceptable amino acid conjugate or salt thereof.

In one of the embodiments, the present application relates to a method of treating or preventing an intestinal inflammatory disease or condition, comprising administering to a subject in need thereof Compound of formula A, or a pharmaceutically acceptable amino acid conjugate or salt thereof. In one embodiment, the present application relates to a method of treating. In one embodiment, the present application relates to a method of preventing. In one embodiment, the intestinal inflammatory disease or condition is loss of intestinal integrity. In one embodiment, the intestinal inflammatory disease or condition is bacterial translocation. In one embodiment, the intestinal inflammatory disease or condition is increased permeability. In one embodiment, the intestinal inflammatory disease or condition is intestinal transplantation. In one embodiment, the intestinal inflammatory disease or condition is intestinal ischemia reperfusion injury (IRI). In one embodiment, the compound of Formula A is administered intravenously (intravenous route of administration). In one embodiment, intravenous route of administration can be used for injections or infusions.

In one of the embodiments, the present application relates to a method of treating or preventing an intestinal inflammatory disease or condition, comprising administering to a subject in need thereof Compound of formula 1, 2, 3, or 4, or a pharmaceutically acceptable amino acid conjugate or salt thereof. In one embodiment, the present application relates to a method of treating. In one embodiment, the present application relates to a method of preventing. In one embodiment, the intestinal inflammatory disease or condition is loss of intestinal integrity. In one embodiment, the intestinal inflammatory disease or condition is bacterial translocation. In one embodiment, the intestinal inflammatory disease or condition is increased permeability. In one embodiment, the intestinal inflammatory disease or condition is intestinal transplantation. In one embodiment, the intestinal inflammatory disease or condition is intestinal ischemia reperfusion injury (IRI). In one embodiment, the compound of Formula 1, 2, 3, or 4 is administered intravenously (intravenous route of administration). In one embodiment, intravenous route of administration can be used for injections or infusions.

In one embodiment the present application relates to a method of treating or preventing an intestinal inflammatory disease or condition, comprising administering to a subject in need thereof Compound of formula 1 or a pharmaceutically acceptable salt thereof. In one embodiment the present application relates to a method of treating an intestinal inflammatory disease or condition, comprising administering to a subject in need thereof Compound of formula 1 or a pharmaceutically acceptable salt thereof. In one embodiment the present application relates to a method of preventing an intestinal inflammatory disease or condition, comprising administering to a subject in need thereof Compound of formula 1 or a pharmaceutically acceptable salt thereof. In one embodiment, the intestinal inflammatory disease or condition is loss of intestinal integrity. In one embodiment, the intestinal inflammatory disease or condition is bacterial translocation. In one embodiment, the intestinal inflammatory disease or condition is increased permeability. In one embodiment, the intestinal inflammatory disease or condition is intestinal transplantation. In one embodiment, the intestinal inflammatory disease or condition is intestinal ischemia reperfusion injury (IRI). In one embodiment the present application relates to a method of treating or preventing intestinal ischemia reperfusion injury (IRI), comprising administering to a subject in need thereof Compound of formula 1 or a pharmaceutically acceptable salt thereof. In one embodiment, the compound of Formula 1 or a pharmaceutically acceptable salt thereof is administered intravenously (intravenous route of administration). In one embodiment, intravenous route of administration can be used for injections or infusions.

The present application also relates to use Compound of formula A, or a pharmaceutically acceptable amino acid conjugate or salt thereof, in the manufacture of a medicament for treating or preventing an intestinal inflammatory disease or condition. In one embodiment, the intestinal inflammatory disease or condition is loss of intestinal integrity. In one embodiment, the intestinal inflammatory disease or condition is bacterial translocation. In one embodiment, the intestinal inflammatory disease or condition is increased permeability. In one embodiment, the intestinal inflammatory disease or condition is intestinal transplantation. In one embodiment, the intestinal inflammatory disease or condition is intestinal ischemia reperfusion injury (IRI). In one embodiment, the medicament comprising compound of Formula A is an injectable. In one of the embodiments, the injectable medicament is for injections or infusions.

The present application also relates to use of Compound of formula A, or a pharmaceutically acceptable amino acid conjugate or salt thereof, in treating or preventing an intestinal inflammatory disease or condition. In one embodiment, the intestinal inflammatory disease or condition is loss of intestinal integrity. In one embodiment, the intestinal inflammatory disease or condition is bacterial translocation. In one embodiment, the intestinal inflammatory disease or condition is increased permeability. In one embodiment, the intestinal inflammatory disease or condition is intestinal transplantation. In one embodiment, the intestinal inflammatory disease or condition is intestinal ischemia reperfusion injury (IRI).

In one of the embodiments, the Compound of formula A is Compound 1, 2, 3, or 4, or a pharmaceutically acceptable amino acid conjugate or salt thereof. In one embodiment Compound of formula A is Compound 1. In one embodiment Compound of formula A is pharmaceutically acceptable salt of Compound 1 (e.g., 1-Na or 1-TEA). In one embodiment Compound of formula A is Compound 2. In one embodiment Compound of formula A is Compound 3. In one embodiment Compound of formula A is Compound 4.

### EXAMPLES

### Example 1. Synthesis of Compounds of the Present Application

Compounds of the present application can be prepared by methods known in the art (e.g., those described in U.S. Pat. Nos. 7,138,390; 7,994,352; 7,932,244; 8,114,862; 9,611,289; 9,777,038; and 10,202,414). For example, a compound of the present application can be prepared by a process as shown in Scheme 1 and disclosed in WO 2014/066819.

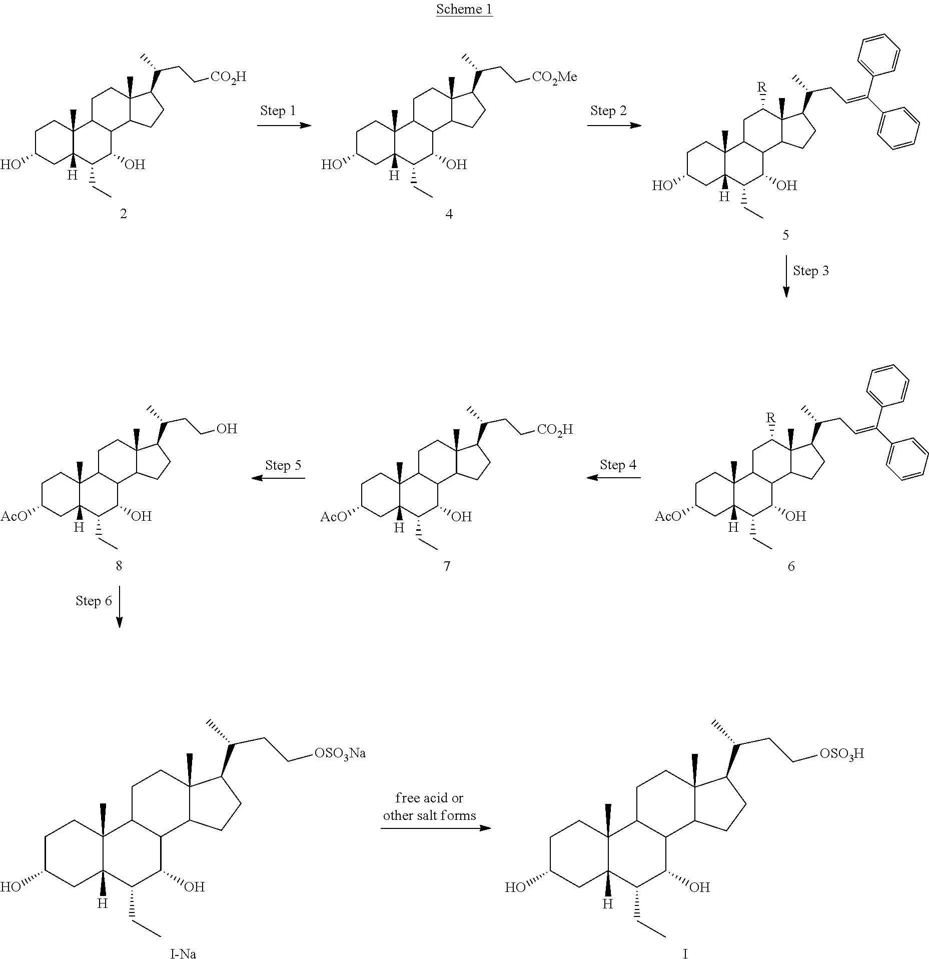

Step 1 is the esterification of Compound 2 to obtain Compound 4. Step 2 is a reaction to form Compound 5 from Compound 4. Step 3 is the protection of the hydroxy group at the C3 position of Compound 5 to afford Compound 6. Step 4 is the oxidative cleavage of Compound 6 to afford Compound 7. Step 5 is the reduction of Compound 7 to afford Compound 8. Step 6 is the sulfonation of Compound 8 to afford the sodium salt of Compound 1 (1-Na). The sodium salt of Compound 1 can be converted to its free acid form (i.e., Compound 1) or other salt forms (e.g., Compound 1-TEA or the triethylammonium salt of Compound 1) according to procedures known in the art.

### Example 2. Reduction of Intestinal Ischemia Reperfusion Injury in a Rat Model Using Compound 1 (INT-767), an FXR/TGR5 Agonist

The study was designed to demonstrate that (1) INT-767 given via intravenous route was more powerful than INT-747 via oral route as a pre-treatment for ischemia reperfusion injury; (2) IV treatment using INT-767 decreased ischemia reperfusion injury by (a) stimulating the FXR receptor to inhibit pro-inflammatory cytokine release and reduce intestinal permeability; (b) stimulating the TGR5 receptor, which inhibited monocytes from producing pro-inflammatory cytokines and stimulating intestinal recovery through GLP-1 upregulation.

### Experimental Design

The experiment was conducted under general anesthesia using intraperitoneal injections of Ketamine/Xylazine:
Median laparotomy with dissection and clamping of the superior mesenteric artery using a microvascular clip. Intestinal ischemia was confirmed by paleness of the small bowel and lack of pulsation in the mesenteric vessels.
Temporary closure of the abdomen using clips
After 60 minutes of warm ischemia, the clamp was removed, 1 cc NaCl 0.9% was administered intraperitoneally (compensating for fluid loss) and the abdomen and skin were closed (using Prolene 3.0 and 4.0 respectively).
At fixed time points after reperfusion (1 hour and 7 days) the animal was anesthetized and euthanized by exsanguination, in order to collect blood- and intestinal samples.
Postoperative analgesia was provided using buprenorphine in the 7-day group
Samples from the ileum were collected and immediately mounted on an Ussing chamber (Verbeke, et al. Obeticholic acid, a farnesoid X receptor agonist, improves portal hypertension by two distinct pathways in cirrhotic rats. Hepatology, 2014; 59(6):2286-2298) at 37° C. in the 1-hour reperfusion group.
In the treatment arms, INT-767 was administered via peripheral venous injection. The controls received an equivalent volume of vehicle only (physiologic saline NaCl 0.9%). INT-747 was administered via oral gavage, dissolved in methylcellulose 1%.
In the sham group, the identical procedure was performed except without occlusion of the superior mesenteric artery.
   Phase 1: Proof of concept of efficacy of IV pre-treatment using INT-767 (N=18 rats, 6 per group), 1 hour of reperfusion group only
Group I: INT-747 30 mg/kg (obeticholic acid) administered orally 24 and 4 hours before start of ischemia
Group II: INT-767 10 mg/kg administered IV 24 and 4 hours before start of ischemia
Group III: INT-767 20 mg/kg administered IV 24 and 4 hours before start of ischemia

### Initial Endpoints:

Intestinal permeability: using the Ussing chamber.
Plasma markers: D/L-Lactate, I-FABP, Villin-1
Histology: Park-Chiu score⁴ with determination of villus length
   Phase 2: Treatment of ischemia reperfusion injury (IV treatment using INT-767) (N=48 rats, 16 per group),
Group I: INT-767 (either 10 or 20 mg/kg) administered IV 5 minutes after start of ischemia
   1 hour after reperfusion: 6 rats
   7 day survival: 10 rats
Group II: Vehicle administered IV 5 minutes after start of ischemia
   1 hour after reperfusion: 6 rats
   7 day survival: 10 rats
Group III: Sham operated rats
   1 hour after reperfusion: 6 rats
   7 day survival: 10 rats

### Endpoints:

Intestinal permeability: In the 1 hour reperfusion group
Ussing chamber experiments: biopsy specimens to be mounted in modified 3 ml
Ussing chambers to measure the trans-epithelial electrical resistance and dextrane passage. The permeability to be correlated to the villus length.
Survival: Survival to be assessed in the 7 day group.
Plasma:
L-Lactate (Blood gas analyzer, ABL-815, Radiometer, Denmark)
D-Lactate (EnzyChrom™ D-Lactate Assay Kit, BioAssay systems)
I-FABP (Proteintech Europe)
Villin 1 levels (Vil 1 Elisa, Cloud Clone)
Measurement by qRT-PCR:
Pro- and anti-inflammatory cytokines (IL-1B, IL-6, TNF-a, INF-y, IL-10 and IL-13)
FXR and small heterodimeric partner (SHP) (Measurement of FXR activation and downstream effect)

### Measurement of Plasma Endotoxin Level: Limulus Amebocyte Lysate (LAL) (Hycult Biotech) Evaluation of Histopathological Changes:

Formaldehyde fixation of the ileum
Scoring system: Park—Chiu and measurement of villus length

### Rationale for Route of Admission:

In this model a peripheral intravenous injection was used. There was a twofold reason for this choice. First, the aim was to achieve a high peak dose necessary to counter the acute onset, inflammation typical for intestinal ischemia reperfusion injury. This differs from other subacute models/chronic studies involving INT-767 (McMahan, et al. Bile acid receptor activation modulates hepatic monocyte activity and improves nonalcoholic fatty liver disease. J Biol Chem. 2013; 288(17):11761-11770; Baghdasaryan, et al. Dual farnesoid X receptor/TGR5 agonist INT-767 reduces liver injury in the Mdr2−/− (Abcb4−/−) mouse cholangiopathy model by promoting biliary HCO3- output. Hepatology. 2011; 54(4):1303-1312; R₁ zzo, et al. Functional characterization of the semisynthetic bile acid derivative INT-767, a dual farnesoid X receptor and TGR5 agonist. Mol. Pharmacol. 2010; 78(4):617-630). Secondly, this route and timing of administration is the most practical when considering the clinical setting in which intestinal ischemia reperfusion injury occurs. This disease occurs unannounced, progresses rapidly and patients go into paralytic ileus for many days. All this makes IV treatment the most ideal route of admission in clinical practice.

### Dosing Rationale:

The initial candidate dosing is based on the limited data available on the subject (R₁ zzo, et al. Mol. Pharmacol. 2010; 78(4):617-630; Roda, et al. Semisynthetic bile acid FXR and TGR5 agonists: physicochemical properties, pharmacokinetics, and metabolism in the rat. J. Pharmacol. Exp. Ther. 2014; 350(1):56-68). The study demonstrated that IV treatment is possible and has similar metabolic effects compared to enteral administration.

To establish the optimal dose, phase 2 potential doses (10 and 20 mg/kg) were planned to be employed. Once the optimal dosage is established, the study could proceed to phase II. In phase I, INT-747 (obeticholic acid) at 30 mg/kg according to our previous protocol was required.
Maximum weight of a rat=350 grams
Phase I: 6 rats using INT 747 (2*30 mg/kg)=150 mg, 12 rats using INT 767 (2*10 and 20 mg/kg)=150 mg
Phase II: Assuming that 20 mg/kg will be the optimal dosage: 16 treated rats=250 mg

### Study Objectives

Primary Endpoint: Intestinal Permeability:
   The permeability of the ileum to be measured by electrical resistance analysis using an Ussing chamber.
Secondary Endpoint(s):
   Survival: In the 7 day group, survival in days to be recorded
   Histological scoring: Park/Chiu score⁴ (0-8) and villus length
   Bacterial translocation: Lipopolysaccharide measurement (ELISA) as surrogate of bacterial sepsis (Opal, et al. Relationship between plasma levels of lipopolysaccharide (LPS) and LPS-binding protein in patients with severe sepsis and septic shock. J Infect Dis. 1999; 180(5):1584-1589).
   Plasma biomarkers: L-lactate (Nielsen et al., L- and d-lactate as biomarkers of arterial-induced intestinal ischemia: An experimental study in pigs. Int J Surg. 2012; 10(6):296-300) (blood gas analysis), Vil-1 (ELISA), D-Lactate (ELISA), I-FABP (Thuijls et al., Early Diagnosis of Intestinal Ischemia Using Urinary and Plasma Fatty Acid Binding Proteins. Ann Surg. 2011; 253(2):303-308) (Western Blot)
   Inflammatory cytokines: IL-1β and TNFα (qPCR), IL-6 (ELISA),
   Anti-inflammatory cytokines: IL-10, IL-13 (qPCR)
   GLP 1 levels: both mucosal and plasma active GLP-1 (ELISA)

### Duration:

Based on the previous experience using this model, the timeline was:
Estimated duration (months) from end of study to completion of study report: 3 months;
Estimated duration (months) from end of study to submission of manuscript (if applicable) 12 months.

### Materials and Methods

In a validated rat model (Ceulemans, et al., Farnesoid-X Receptor Activation Attenuats Intestinal Ischemia Reperfusion Injury in Rats. PLoS One (2017) 12(1): e0169331) (Sprague-Dawley, male, 300 g) of intestinal IRI (laparotomy and clamping of superior mesenteric artery), 3 groups (n=6/group) were investigated: i/Sham (only laparotomy); ii/Ischemia 60 min+reperfusion 60 min (IR)+intravenous vehicle; iii/Ischemia 60 min+reperfusion 60 min+intravenous FXR/TGR5-agonist (IR+FXR/TGR5). Animals were sacrificed by exsanguination under anesthesia. For each group, 10 additional animals were included for a 7-day survival analysis. It has been determined that individual group we would require 6 rats per group to detect a significant difference in permeability.

Model (Ceulemans, et al. 2017): Animals were anaesthetized by an intraperitoneally administered mix of ketamin (1*100 mg/kg, Anesketin, Eurovet, the Netherlands) and xylazin (1*10 mg/kg, Xyl-M 2%, Van Miert&Dams Chemie, Belgium). In accordance to animal welfare, rats were monitored at least 3 times daily and buprenorphine (Vetergesic) was used for analgesia during the first 2 days following the experiments. A morbidity score (including weight changes: 3 points, behavior: 3 points and stool presence: 1 point) with a maximum of 7 was used. If a score was higher than 3, the protocol included euthanasia by overdose of pentobarbital (Nembutal) after anesthesia induction. Intestinal IRI was induced after median laparotomy by isolated temporary clamping of the superior mesenteric artery. This is a well-validated model of intestinal IRI and very often used in literature due to its ‘minimal-touch’ technique and clinical significance. 60 minutes of ischemia were chosen since this time period provokes far more deleterious effects of intestinal ischemia than 30 or 45 minutes and keeps the animal alive during the reperfusion period.

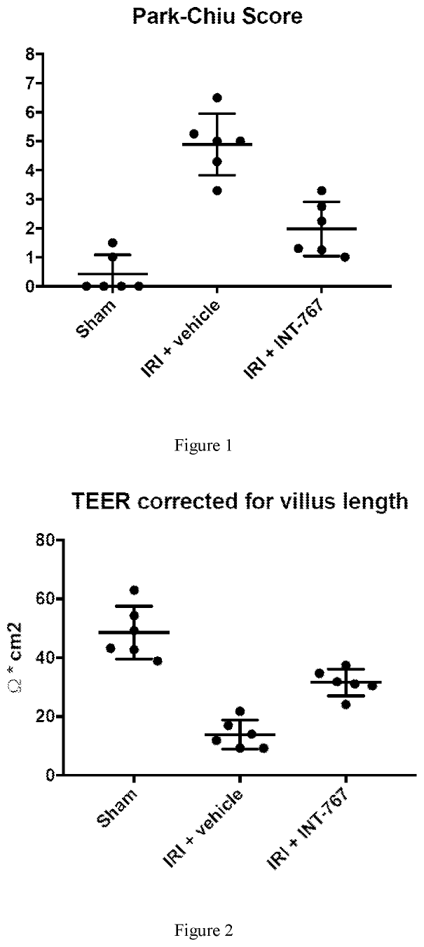

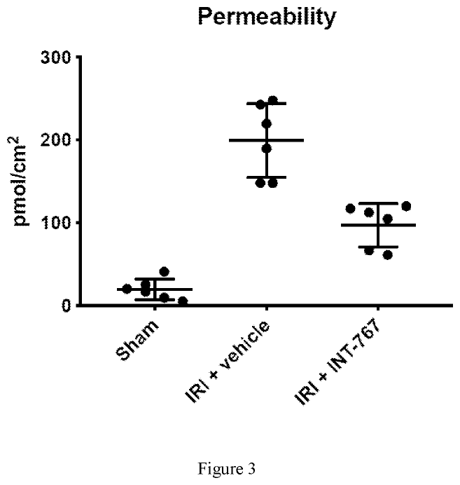

FXR/TGR5-agonist INT-767 (Intercept Pharma, USA) or vehicle only was administered intravenously in a single dose at 10 mg/kg, 15 minutes after start of ischemia. Analyzed endpoints: 1/Histology: Park/Chiu score and villus length (FIG. 1); 2/Permeability (transepithelial electrical resistance (TEER) (FIG. 2); Ussing chamber and FD20 translocation measurements) (FIG. 3); 3/Inflammatory cytokines: IL-6 (ELISA), IL-113 and TNFα (qPCR); and 4/Anti-inflammatory cytokines: IL-10, IL-13 (qPCR). Applied statistics were: One-way Anova and post-hoc Bonferroni (normal variance, within group comparison) and Kaplan-Meier log-rank analysis (survival); p<0.05 was considered significant (GraphPad v8.0, La Jolla, Calif., USA).

### Results

In this model, intestinal IRI led to pronounced damage resulting in high Park/Chiu scores and decreased villus length (FIG. 1). The observed PC score showed that the intestinal histology is partially preserved using Compound 1(INT-767) treatment. The resulting intestinal permeability led to increased inflammatory cytokines expression. As shown in FIG. 1 (the histological damage score (Park-Chiu) of treated and untreated rats; compared to sham subjects), treatment with Compound 1 (INT-767) significantly protects the intestine against IRI-related damage. INT-767 treatment significantly reduced these alterations. Both markers for intestinal permeability (e.g., TEER and FD20) were improved (i.e., reduction of IRI induced epithelial damage). Both, electrical resistance (TEER) and translocation (FD 20) were significantly reduced (FIGS. 2 and 3). As shown in FIG. 2, Compound 1 (INT-767) reduced damage to the intestine leading to increased TEER compared to vehicle treatment. Compound 1 (INT-767) reduces damage to the intestine leading to reduced permeability compared to vehicle treatment (FIG. 3). 7-day survival was improved significantly after treatment. Results are summarized in the Table 1. For the first time, it has been demonstrated that treatment with a dual FXR/TGR5 agonist significantly decreased damage caused by intestinal IRI. These results show that FXR and TGR5 receptors are promising targets for intestinal graft protection. The ability to administer this substance intravenously greatly enhances the potential applicability for the frequent pathology of intestinal infarction as well as for transplantation.

TABLE 1
Analysis of different endpoints after Farnesoid-X receptor (FXR)
and Takeda G-protein-coupled receptor 5 (TGR5) agonist treatment
in a model of intestinal ischemia reperfusion injury

| Endpoints | | | | P-value |
|---|---|---|---|---|
| Median | | IRI + | IRI + | (IRI + 767 vs |
| (range) | SHAM | Vehicle | INT-767 | IRI + vehicle) |
| Park/Chiu | 0 (0-1.0) | 5.0 (3.3- | 1.8 (1.0- | P = 0.0005 |
| (0-8) | | 6.5) ^{###} | 3.3) ^{***} | |
| Villus length | 273 | 104 (66- | 201 (168- | P = 0.0001 |
| (μm) | (205-286) | 118) ^{###} | 280) ^{***} | |
| TEER | 49 (39-63) | 14 (9-21) ^{###} | 32 (24- | P < 0.0001 |
| (Ohm*cm²) | | | 37) ^{***} | |
| (Villus length | | | | |
| corrected) | | | | |
| FD 20 | 18.5 | 204.7 | 108.5 (61.1- | P = 0.0007 |
| Permeability | (5.3-40.8) | (147.9- | 119.8) ^{***} | |
| (pmol/cm²) | | 247.9) ^{###} | | |
| IL-6 | | 207.4 (148.3- | 155.5 (8.7- | P = 0.0492 |
| (fold change) | | 403.1) | 181.7) ^{*} | |
| IL-1-β | | 7.8 (5.0-13.8) | 3.5(1.9- | P = 0.0140 |
| (fold change) | | | 11.1) ^{*} | |
| TNF-α | | 6.9 (2.4-9.9) | 3.6 (2.7- | P = 0.0019 |
| (fold change) | | | 4.4) ^{**} | |
| IL-10 | | 10.7 (5.4- | 16.6 (11.2- | P = 0.0257 |
| (fold change) | | 14.6) | 21.0) ^{*} | |
| IL-13 | | 11.4(4.2- | 17.0 (11.6- | P = 0.0139 |
| (fold change) | | 15.9) | 20.4) ^{*} | |
| 7-day | 100% | 0% | 50% ^{*} | |
| survival (%) | | | | |

Legend:
FD 20 = fluorescein isothiocyanate-labelled 20 kiloDalton dextran;
TEER = Trans Epithelial Electrical Resistance;
IR − FXR/TGR5: 60 min of ischemia and 60 min of reperfusion without FXR/TGR 5-agonist treatment;
IR + FXR/TGR5: 60 min of ischemia and 60 min of reperfusion with FXR/TGR5-agonist treatment;
^{*} IRI + Vehicle vs IRI + INT-767: ^{*} P < 0.05, ^{**} < 0.01, ^{***}: P < 0.001.
^{#} IRI + Vehicle vs SHAM ^{#} P < 0.05, ^{##} P < 0.01, ^{###} P < 0.001.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A method of treating an intestinal inflammatory disease or condition in a subject in need thereof, comprising administering to the subject a therapeutically effective amount of a compound of Formula A: 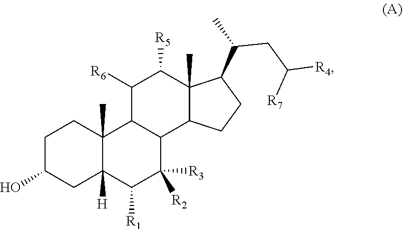
or a pharmaceutically acceptable salt or amino acid conjugate thereof, wherein:
R₁ is C₁-C₆ alkyl;
R₂, R₃, R₅, and R₆ are each independently H or OH;
R₄ is CO₂H or OSO₃H; and
R₇ is H or C₁-C₆ alkyl.

2. The method of claim 1, comprising administering to the subject a compound of formula 1: 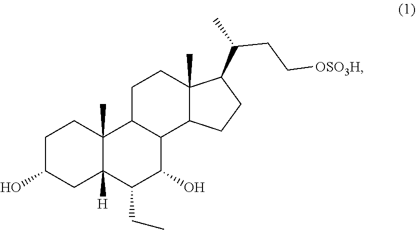
or a pharmaceutically acceptable salt thereof.

3. The method of claim 1 or 2, comprising administering to the subject a compound of formula 1-Na:

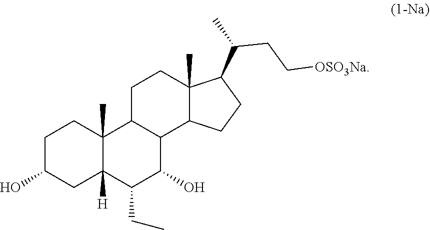

4. The method of claim 1 or 2, comprising administering to the subject a compound of formula 1-TEA:

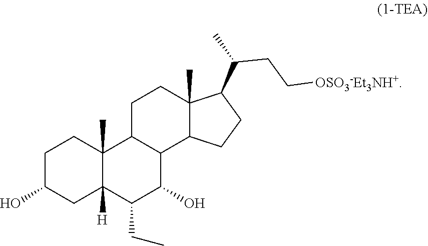

5. The method of any one of the preceding claims, wherein the disease or condition is modulated by FXR.

6. The method of any one of the preceding claims, wherein the disease or condition is modulated by TGR5.

7. The method of any one of the preceding claims, wherein the disease is intestinal ischemia reperfusion injury.

8. A method of reducing intestinal permeability in a subject in need thereof, comprising administering to the subject a therapeutically effective amount of a compound of Formula A: 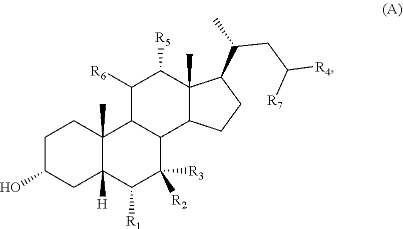
or a pharmaceutically acceptable salt or amino acid conjugate thereof, wherein:
R₁ is C₁-C₆ alkyl;
R₂, R₃, R₅, and R₆ are each independently H or OH;
R₄ is CO₂H or OSO₃H; and
R₇ is H or C₁-C₆ alkyl.

9. A method of stimulating intestinal recovery through GLP-1 upregulation in a subject in need thereof, comprising administering to the subject a therapeutically effective amount of a compound of Formula A: 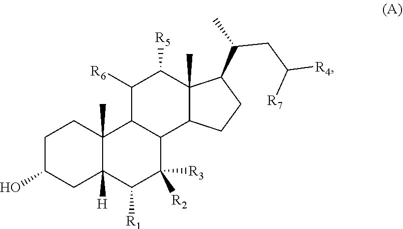
or a pharmaceutically acceptable salt or amino acid conjugate thereof, wherein:
R₁ is C₁-C₆ alkyl;
R₂, R₃, R₅, and R₆ are each independently H or OH;
R₄ is CO₂H or OSO₃H; and
R₇ is H or C₁-C₆ alkyl.
